(19) United States
(12) Patent Application Publication
(10) Pub. No.: US 2025/0288725 A1
(43) Pub. Date: Sep. 18, 2025

(54) SYSTEMS, METHODS, AND DEVICES OF EXOSOME DELIVERY FOR FILLING BONE FRACTURE VOIDS

(71) Applicant: THORAGENIX INNOVATIONS, INC., Boca Raton, FL (US)

(72) Inventors: Frederick J. THABET, Boca Raton, FL (US); Ravi KANAGALA, Boca Raton, FL (US); Keri GEORGE, Calgary (CA)

(73) Assignee: THORAGENIX INNOVATIONS, INC., Boca Raton, FL (US)

(21) Appl. No.: 18/861,511

(22) PCT Filed: Apr. 27, 2023
(86) PCT No.: PCT/US2023/066289

§ 371 Date: Oct. 29, 2024

Related U.S. Application Data
(60) Provisional application No. 63/337,027, filed on Apr. 29, 2022.

(51) Int. Cl. A61L 27/38; A61L 27/54

(52) U.S. Cl. CPC A61L 27/3821; A61L 27/3808; A61L 27/3817; A61L 27/3834; A61L 27/54; A61L 2300/258; A61L 2400/06; A61L 2430/02; A61L 2430/06

(57) ABSTRACT

Systems, methods, and devices include techniques for healing a bone void and/or regenerating a portion of bone by forming an exosome solution with one or more cell-derived exosomes and one or more cellular components. The one or more cellular components include at least one of bone growth regenerating cellular components or cartilage regeneration components, such as bone regeneration cells, marrow regeneration cells, vessel regeneration cells, muscle regeneration cells, and/or combinations therein. Systems disclosed herein also include an exosome carrier material such as a liquid, paste, or gel. The exosome carrier material can be inserted or injected into a bone void (e.g., a rib facture or a thoracic fracture) to instigate healing at the bone void.

### CROSS-REFERENCE TO RELATED APPLICATIONS

This application is a continuation-in-part of and claims priority to U.S. Provisional Patent Application No. 63/337,027, filed Apr. 29, 2022, and titled “Systems, Methods, and Devices of Exosome Delivery for Filling Bone Fracture Voids,” the entirety of which is incorporated herein by reference.

### BACKGROUND

Various bone voids can occur from injury, surgery, or other sources of damage to the human body. Bone void injuries can occur in remote locations with limited medical access and, in other instances, bone void injuries are treated in clinical settings. Additionally, nonunion with bone defects, a common complication after long bone fracture, is a major challenge for orthopedic surgeons worldwide because of the high incidence rate and difficulties in achieving successful treatment. Bone voids and bone defects are the main complications of nonunion. The conventional biological treatments for nonunion with bone defects involve the use of autologous bone grafts or bone graft substitutes. Traditional nonunion treatments have always been associated with safety issues and various other complications.

### SUMMARY

Implementations described and claimed herein can address the foregoing problems by providing systems, methods, and devices for healing a bone void. For instance, method(s) can include forming an exosome carrier material including one or more cell-derived exosomes and one or more cellular components; inserting the exosome carrier material into a bone void; and/or instigating, with the one or more cell-derived exosomes and the one or more cellular components, healing at the bone void. The one or more cellular components can include one or more bone regenerating components or one or more cartilage regenerating components. Furthermore, the one or more bone regenerating components can include one or more of an osteoclast cell, an endothelial cell, a stem cell, or a macrophage cell. Additionally, the one or more cartilage regenerating components can include one or more of a chondrite, a fibroblast, or a platelet.

In some scenarios, the one or more cellular components can include a bone cellular component, a muscle cellular component, a marrow cellular component, or a vessel cellular component. Moreover, the one or more cellular components can include a mesenchymal stem cell and a macrophage as marrow regenerating components. The one or more cellular components can include an endothelial cell as a vessel regenerating component, or a myocyte as a muscle regenerating component. Additionally or alternatively, the bone void can be a thoracic fracture; and/or inserting the exosome carrier material into the bone void can include injecting the exosome carrier material into the thoracic fracture.

In some examples, a method of healing a bone void includes forming an exosome solution including one or more cell-derived exosomes and one or more bone growth generating cellular components; forming an exosome carrier material using at least some of the exosome solution; providing the exosome carrier material into a bone void; and/or instigating, with the one or more cell-derived exosomes and the one or more bone growth generating cellular components, healing in the bone void. The bone void can be a rib fracture, the exosome carrier material can be a liquid or a gel, and/or providing the exosome carrier material into the bone void can include injecting the exosome carrier material into the rib fracture.

In some examples, forming the exosome carrier material using at least some of the exosome solution includes injecting the exosome solution into a sealed container while a graft is in the sealed container. The method can further include compressing a portion of bone around the bone void. The bone void can be a thoracic fracture; and/or an amount of the exosome carrier material can correspond to a size of the thoracic fracture. The one or more bone growth generating cellular components can include one or more of an osteoclast, an endothelial cell, a stem cell, and/or a macrophage. The exosome solution can further include mesenchymal stem cells as a marrow regeneration component of the exosome solution, and/or a myocyte cell as a muscle regeneration component of the exosome solution. The one or more cell-derived exosomes can include micro ribonucleic acid (miRNA) and protein.

In some scenarios, a method of healing a bone void includes providing a bone void in an operating environment; forming an exosome carrier material including one or more cell-derived exosomes and one or more cellular components, the one or more cellular components including at least one of bone growth regenerating cellular components or cartilage regenerating components; and/or inserting the exosome carrier material into the bone void to instigate healing at the bone void. The one or more cellular components can include an endothelial cell as a vessel regeneration component of the exosome carrier material. Additionally, the bone void can be thoracic fracture; and the exosome carrier material can be a liquid, gel, or paste which at least partially fills the thoracic fracture.

Other implementations are also described and recited herein. Further, while multiple implementations are disclosed, still other implementations of the presently disclosed technology will become apparent to those skilled in the art from the following detailed description, which shows and describes illustrative implementations of the presently disclosed technology. As will be realized, the presently disclosed technology is capable of modifications in various aspects, all without departing from the spirit and scope of the presently disclosed technology. Accordingly, the drawings and detailed description are to be regarded as illustrative in nature and not limiting.

### BRIEF DESCRIPTION OF THE DRAWINGS

The foregoing summary, as well as the following detailed description, will be better understood when read in conjunction with the appended drawings. For the purpose of illustration, there is shown in the drawings certain examples of the disclosed subject matter. It should be understood, however, that the disclosed subject matter is not limited to the precise examples and features shown. The accompanying drawings, which are incorporated in and constitute a part of this specification, illustrate an implementation of systems and methods consistent with the disclosed subject matter and, together with the description, serves to explain advantages and principles consistent with the disclosed subject matter, in which:

FIG. 1 illustrates an example system for healing a bone void with an exosome carrier material;

FIG. 2 illustrates an example system for healing a bone void with an exosome carrier material, which can be combined with or can form a portion of the system(s) depicted in FIG. 1;

FIG. 3 illustrates an example system for healing a bone void with a graft, which can be combined with or can form a portion of the system(s) depicted in FIGS. 1 and 2;

FIG. 4 illustrates an example method of healing a bone void with an exosome carrier material, which can be performed by any of the system(s) depicted in FIGS. 1-3;

FIG. 5 illustrates an example method of healing a bone void with an exosome carrier material, which can be performed by any of the system(s) depicted in FIGS. 1-3; and

FIG. 6 illustrates an example method of healing a bone void with an exosome carrier material, which can be performed by any of the system(s) depicted in FIGS. 1-3.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the examples described herein. However, it will be understood by those of ordinary skill in the art that the examples described herein can be practiced without these specific details. In other instances, methods, procedures and components have not been described in detail so as not to obscure the related relevant feature being described. Also, the description is not to be considered as limiting the scope of the examples described herein. The drawings are not necessarily to scale and the proportions of certain parts may be exaggerated to better illustrate details and features of the present disclosure.

Further, as the presently disclosed technology is susceptible to examples of many different forms, it is intended that the present disclosure be considered as an example of the principles of the presently disclosed technology and not intended to limit the presently disclosed technology to the specific examples shown and described. Any one of the features of the presently disclosed technology may be used separately or in combination with any other feature. References to the terms “example,” “examples,” and/or the like in the description mean that the feature and/or features being referred to are included in, at least, one aspect of the description. Separate references to the terms “example,” “examples,” and/or the like in the description do not necessarily refer to the same example and are also not mutually exclusive unless so stated and/or except as will be readily apparent to those skilled in the art from the description. For example, a feature, structure, process, step, action, or the like described in one example may also be included in other examples, but is not necessarily included. Thus, the presently disclosed technology may include a variety of combinations and/or integrations of the examples described herein. Additionally, all aspects of the present disclosure, as described herein, are not essential for its practice. Likewise, other systems, methods, features, and advantages of the presently disclosed technology will be, or become, apparent to one with skill in the art upon examination of the figures and the description. It is intended that all such additional systems, methods, features, and advantages be included within this description, be within the scope of the presently disclosed technology, and be encompassed by the claims.

Systems, methods, and devices disclosed herein use exosomes (e.g., extracellular vesicles) to improve bone healing, for instance, as part of a bone void filling, bone void healing, and/or bone fusing procedure. Exosomes can be derived from placental or umbilical stem cells. Exosomes are the protein enzymatic catalyst of a stem cell that gives the stem cell its ability to speed up the natural process the body goes through to heal. The exosomes can be combined with one or more additive cellular components to improve its bone healing functions (e.g., in a solution, a paste, a gel, a powder, a solid, etc.). The exosomes can be used to heal bone, such as the severed portions of a sternum during a sternum fusing procedure following a sternotomy (e.g., to provide access to the chest cavity for surgery). In some instances, the exosomes can be added to a graft (e.g., an implant formed of natural material, synthetic material, or combinations thereof), and the graft can be inserted and/or compressed between the two severed bone portions to improve bone fusion and healing.

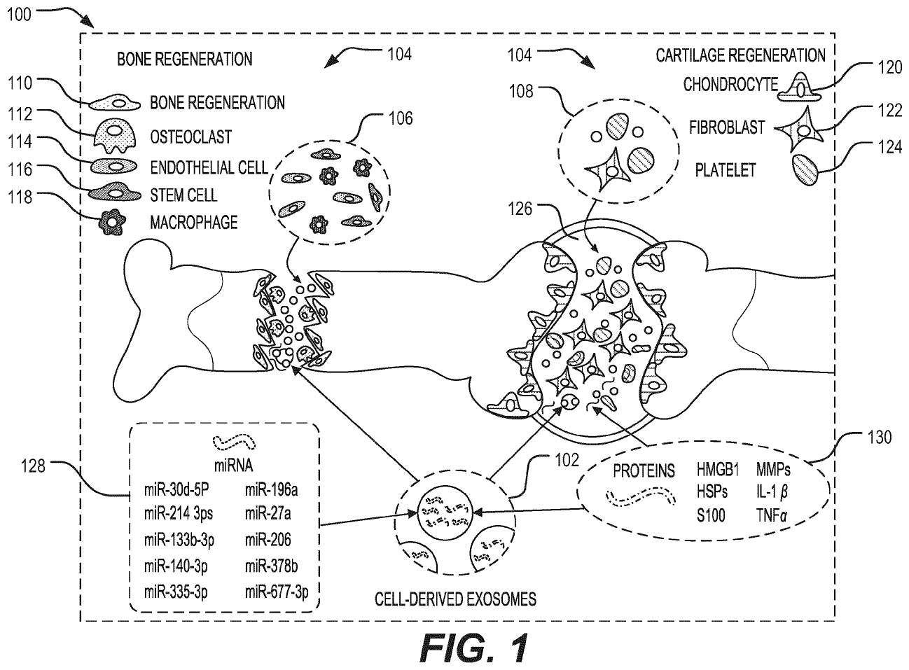

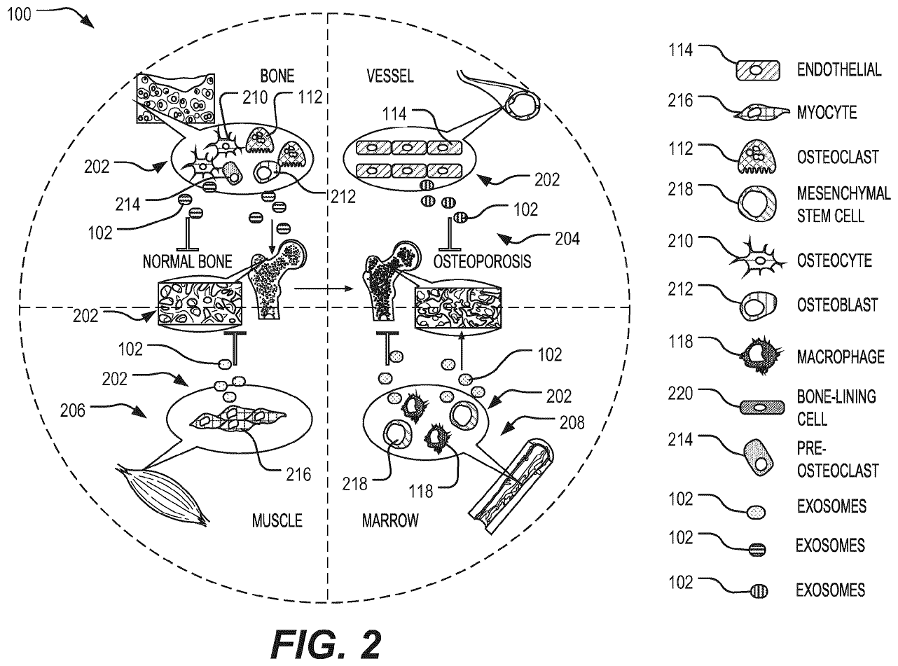

FIGS. 1 and 2 illustrate various components of a bone healing procedure 100 using exosomes 102. For instance, FIG. 1 depicts the cellular components 104 for bone regeneration (e.g., bone regeneration components 106) and/or cartilage regeneration (e.g., cartilage regeneration components 108) which can be combined with and/or used with the exosomes 102. The bone regeneration components 106 can include a bone regeneration cell 110, an osteoclast 112, an endothelial cell 114, a stem cell 116, a macrophage 118, and/or any combination thereof. The cartilage regeneration components 108 can include a chondrite 120, a fibroblast 122, a platelet 124, and/or any combination thereof. In some examples, the cartilage regeneration components 108 can include or be immersed in a synovial fluid 126. Moreover, the cell-derived exosomes 102 can include (e.g., contain) miRNA 128 (e.g., miR-30d-5P; miR-214 3ps; miR-133b-3p; miR-140-3p; miR-335-3p; miR-196a; miR-27a; miR0206; miR-378 bp; or miR-677-3p). Additionally or alternatively, the exosomes 102 can include (e.g., contain) one or more proteins 130 (e.g., HMGB1, HSPs, S100, MMPs, IL-1β; or TNFα).

FIG. 2 depicts cellular components 202 such as bone regeneration components 106, vessel regeneration components 204, muscle regeneration components 206, and/or marrow regeneration components 208, which can form part of the exosome-cellular component solution 312. The bone regeneration components 106 can include the osteoclast 112, an osteocyte 210, an osteoblast 212, a pre-osteoclast 214, and/or combinations thereof. The vessel regeneration components 204 can include an endothelial cell 114, which can be used in a exosome-cellular component solution 312 with cellular components 202 designed for osteoporosis patients. The muscle regeneration components 206 can include a myocyte 216. The marrow regeneration components 208 can include a mesenchymal stem cell 218 and/or a macrophage 118. The cellular components 202 can also include a various other bone-lining cells 220. As shown in FIG. 2, exosomes 102 can be used with any combinations of the cellular components 202 (e.g., the bone regeneration components 106, the vessel regeneration components 204, the muscle regeneration components 206, and/or the 208) to regenerate marrow of the bone (e.g., during a sternum bone fusing procedure) and/or other cellular bone structures.

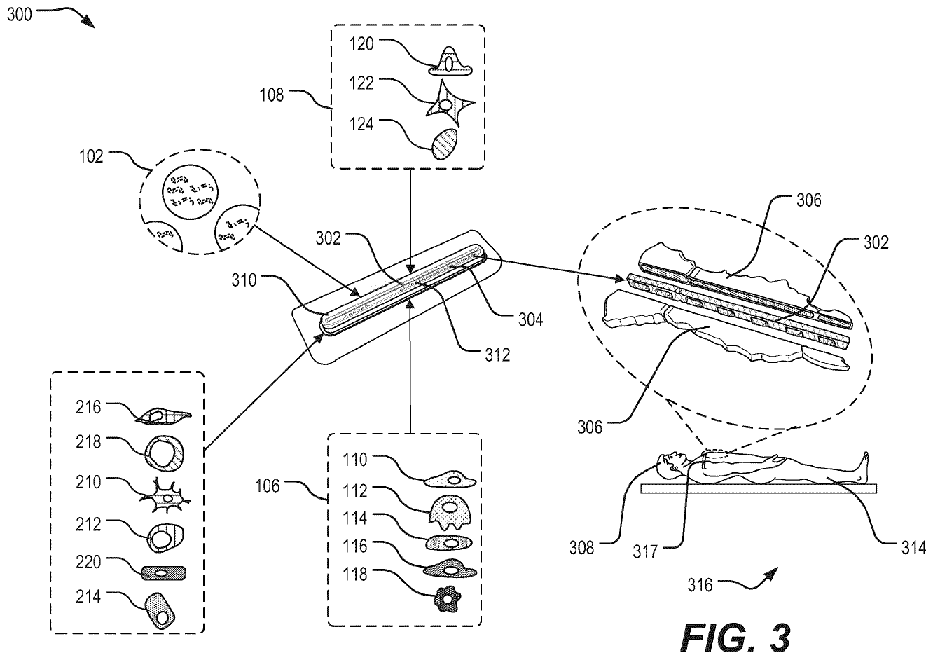

FIG. 3 illustrates an example system 300 which can be combined with or form a portion of the bone healing procedure 100 depicted in FIGS. 1 and 2.

In some examples, a graft 302 can be hydrated with the exosomes 102 and/or the cellular components 202 (by soaking the graft 302 directly in exosomes 102 (e.g., an exosomes solution 304) prior to implantation (e.g., between two severed portions of bone 306 of a patient 308). The graft 302 can be soaked in a sealed container 310 (e.g., a sterile container), which can receive an injection of the cellular components 202 and/or the exosomes 102. The sealed container 310 can be opened and the graft 302 can be retrieved from an exosome-cellular component solution 312 in which the graft 302 was soaked prior to placement in the patient 308. Additionally or alternatively, exosomes 102 and/or cellular components 202 can be injected into the graft 302 after implantation. In some examples, a particular combination of cellular components 202 is added to the exosomes solution 304 to form the exosome-cellular component solution 312 to match particular patient characteristics (e.g., osteoporosis, osteomyelitis, scoliosis, age, other diseases, etc.) In some scenarios, a physician can inject exosomes 102 and/or the exosome-cellular component solution 312 directly into the graft 302 itself, or can inject the exosomes 102 and/or the exosome-cellular component solution 312 into the graft 302 via one or more injection ports built into the graft 302. Furthermore, exosomes 102 and/or the cellular components 202 can be used in the construction of the bone graft 302 itself. Infusion of exosomes 102 into the graft 302 can accelerate bone growth once the graft is placed and secured into the body 314 of the patient 308 (e.g., with one or more securement wires).

Additionally, multiple areas of the body 314 can be treated with exosomes 102 and/or by injecting exosomes 102 into the injection port in the graft 302. Any part of the body 314 including a bone, a bone fracture, a bone defect, a bone void, and/or a degenerative bone disorder can be treated with exosomes 102 (e.g., with placement of an exosome-soaked and/or exosome-injected graft 302 at the location in the body 314). The exosomes 102 can be delivered locally at any site. Moreover, as noted above, the delivery system for the exosomes 102 can be delivery via injection, via placing with the bone graft 302, with a collagen carrier (e.g., human and/or xenograft), a matrix (e.g., collagen matrix), and/or via a lyophilization process. Adding exosomes 102 onto the bone and/or into the fracture site can encourage bone growth. Examples that can be treated using an exosome-soaked and/or exosome-injected graft 302 include osteoporosis, osteomyelitis, osteomyelitis, scoliosis, and so forth. This can occur in an operating environment 316, and/or other clinical settings. The graft 302 engorged with the exosomes solution 304 can be provided to the operating environment 316 by packaging the graft 302 in the sealed container 310 and/or sending the graft 302 to a third-party clinical entity (e.g., a hospital, a surgeon, a distributor, etc.). Furthermore, the exosomes 102 can be combined with the cellular components 104/202 in another media, such as a paste, a gel, a rigid block, a solid, a powder, combinations thereof, and the like. The exosomes 102 can be inserted and/or injected (e.g., with a needle or injection gun) into a bone void, such as a thoracic fracture and/or rib fracture 317, to instigate healing at the bone void. Additionally, one or more other medical additives can be included in the exosome-cellular component mixture, such as analgesic, an antibiotic, an adhesive, a blood coagulant, other medications, or combinations thereof.

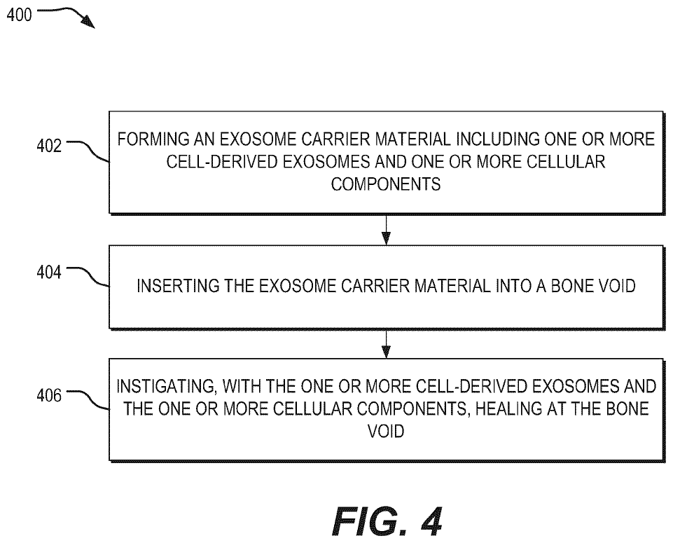

FIGS. 4-6 illustrate example method(s) 400-600 for using exosomes to regenerate a portion of a bone. The method(s) 400-600 can be similar to, identical to, and/or can form a portion of the bone healing procedure 100. Additionally or alternatively, method(s) 400-600 can be performed by the system 300 depicted in FIG. 3.

As depicted in FIG. 4 at operation 402, the method 400 can form an exosome carrier material including one or more cell-derived exosomes and one or more cellular components. At operation 404, the method 400 can insert the exosome carrier material into a bone void. At operation 406, the method 400 can instigate, with the one or more cell-derived exosomes and the one or more cellular components, healing at the bone void.

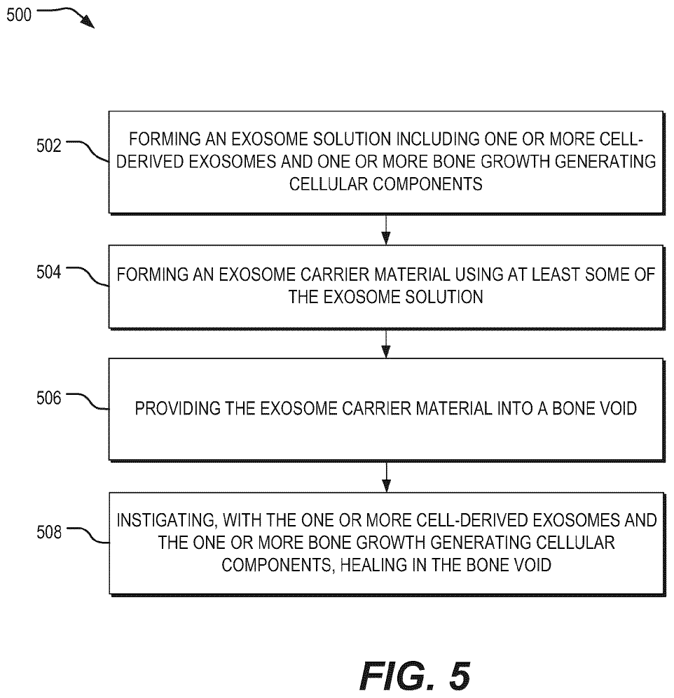

As depicted in FIG. 5, at operation 502, the method 500 can form an exosome solution including one or more cell-derived exosomes and one or more bone growth generating cellular components. At operation 504, the method 500 can form an exosome carrier material using at least some of the exosome solution. At operation 506, the method 500 can provide the exosome carrier material into a bone void. At operation 508, the method 500 can instigate, with the one or more cell-derived exosomes and the one or more bone growth generating cellular components, healing in the bone void

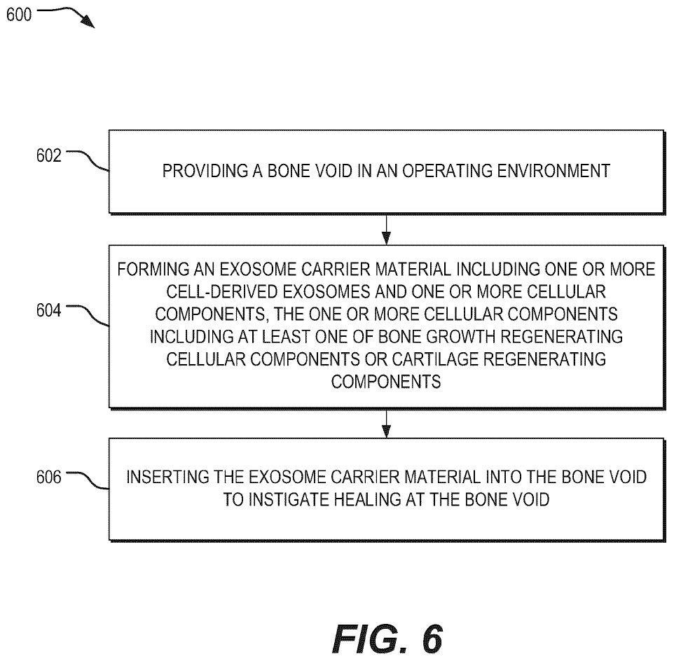

As depicted in FIG. 6, at operation 602, the method 600 can provide a bone void in an operating environment. At operation 604, the method 600 can form an exosome carrier material including one or more cell-derived exosomes and one or more cellular components, the one or more cellular components including at least one of bone growth regenerating cellular components or cartilage regenerating components. At operation 606, the method 600 can insert the exosome carrier material into the bone void to instigate healing at the bone void.

It is to be understood that the specific order or hierarchy of steps in the methods 400-600 and discussed throughout this disclosure are instances of example approaches and can be rearranged while remaining within the disclosed subject matter. For instance, any of the operations discussed in methods 400-600 and throughout this disclosure may be omitted, repeated, performed in parallel, performed in a different order, and/or combined with any other of the operations of methods 400-600 and throughout this disclosure.

While the present disclosure has been described with reference to various implementations, it will be understood that these implementations are illustrative and that the scope of the present disclosure is not limited to them. Many variations, modifications, additions, and improvements are possible. More generally, implementations in accordance with the present disclosure have been described in the context of particular implementations. Functionality may be separated or combined differently in various implementations of the disclosure or described with different terminology. These and other variations, modifications, additions, and improvements may fall within the scope of the disclosure as defined in the claims that follow.

What is claimed is:

1. A method of healing a bone void, the method comprising:
forming an exosome carrier material including one or more cell-derived exosomes and one or more cellular components;
inserting the exosome carrier material into a bone void; and
instigating, with the one or more cell-derived exosomes and the one or more cellular components, healing at the bone void.

2. The method of claim 1,
wherein,
the one or more cellular components include one or more bone regenerating components or one or more cartilage regenerating components.

3. The method of claim 2,
wherein,
the one or more bone regenerating components include one or more of an osteoclast cell, an endothelial cell, a stem cell, or a macrophage cell.

4. The method of claim 2,
wherein,
the one or more cartilage regenerating components include one or more of a chondrite, a fibroblast, or a platelet.

5. The method of claim 1,
wherein,
the one or more cellular components include a bone cellular component, a muscle cellular component, a marrow cellular component, or a vessel cellular component.

6. The method of claim 1,
wherein,
the one or more cellular components include a mesenchymal stem cell and a macrophage as marrow regenerating components.

7. The method of claim 6,
wherein,
the one or more cellular components include an endothelial cell as a vessel regenerating component, or a myocyte as a muscle regenerating component.

8. The method of claim 1,
wherein,
the bone void is a thoracic fracture; and
inserting the exosome carrier material into the bone void includes injecting the exosome carrier material into the thoracic fracture.

9. A method of healing a bone void, the method comprising:
forming an exosome solution including one or more cell-derived exosomes and one or more bone growth generating cellular components;
forming an exosome carrier material using at least some of the exosome solution;
providing the exosome carrier material into a bone void; and
instigating, with the one or more cell-derived exosomes and the one or more bone growth generating cellular components, healing in the bone void.

10. The method of claim 9,
wherein,
the bone void is rib fracture;
the exosome carrier material is a liquid or a gel; and
providing the exosome carrier material into the bone void includes injecting the exosome carrier material into the rib fracture.

11. A method of claim 10, wherein:
forming the exosome carrier material using at least some of the exosome solution includes injecting the exosome solution into a sealed container while a graft is in the sealed container.

12. The method of claim 10, further comprising:
compressing a portion of bone around the bone void.

13. The method of claim 9,
wherein,
the bone void is a thoracic fracture; and
an amount of the exosome carrier material corresponds to a size of the thoracic fracture.

14. The method of claim 9,
wherein,
the one or more bone growth generating cellular components include one or more of an osteoclast, an endothelial cell, a stem cell, or a macrophage.

15. The method of claim 14,
wherein,
the exosome solution further includes mesenchymal stem cells as a marrow regeneration component of the exosome solution.

16. The method of claim 14,
wherein,
the exosome solution further includes a myocyte cell as a muscle regeneration component of the exosome solution.

17. The method of claim 9,
wherein,
the one or more cell-derived exosomes includes micro ribonucleic acid (miRNA) and protein.

18. A method of healing a bone void, the method comprising:
providing a bone void in an operating environment;
forming an exosome carrier material including one or more cell-derived exosomes and one or more cellular components, the one or more cellular components including at least one of bone growth regenerating cellular components or cartilage regenerating components; and
inserting the exosome carrier material into the bone void to instigate healing at the bone void.

19. The method of claim 18,
wherein,
the one or more cellular components include an endothelial cell as a vessel regeneration component of the exosome carrier material.

20. The method of claim 19,
wherein,
the bone void is a thoracic fracture; and
the exosome carrier material is a liquid, gel, or paste which at least partially fills the thoracic fracture.
